(19) United States
(12) Patent Application Publication
(10) Pub. No.: US 2007/0111318 A1
(43) Pub. Date: May 17, 2007

(54) Brexetoxin derivative, production process thereof and method for detecting shellfish neurotoxin using same

(76) Inventor: Hitoshi Ishida, Shizuoka-shi (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 10/580,794

(22) PCT Filed: Nov. 26, 2004
(86) PCT No.: PCT/JP04/17598

§ 371 Date: May 26, 2006

(30) Foreign Application Priority Data
Nov. 28, 2003 (JP) 2003-399683

(51) Int. Cl. G01N 33/00; C07D 493/22

(52) U.S. Cl. 436/93; 549/275

(57) ABSTRACT

A novel shellfish neurotoxin is provided. The present invention provides a novel shellfish neurotoxin represented by formula (I), a method for synthesizing that component, and a method for detecting the principal component of that shellfish neurotoxin to investigate the production of shellfish neurotoxin by using that shellfish neurotoxin component.

### TECHNICAL FIELD

The present invention relates to a shellfish neurotoxin present in toxic shellfish, a production process thereof, and a method for detecting shellfish neurotoxin using that shellfish toxin.

### BACKGROUND ART

Food poisoning caused by shellfish neurotoxin following ingestion of bivalves occurred from December 1992 to March 1993 in New Zealand. This food poisoning occurred due to ingestion of shellfish exposed to the red tide of the dinoflagellate, Gymnodinium (G.) breve, and is known as neurotoxic shellfish poisoning (NSP) because of the characteristic neurological symptoms that appear in patients (Non-Patent Documents 1 and 2). Neurotoxic shellfish poisoning occurs following the collection and consumption of shellfish by humans as a result of toxic components produced by special plankton in the ocean being incorporated by shellfish, and brevetoxins such as PbTx-2, PbTx-3, BTX-B1, BTX-B2, BTX-B3 and BTX-B4 have been identified as these shellfish neurotoxins (Non-Patent Documents 3, 4, 5 and 6). However, since the isolation of toxic compounds such as brevetoxins from shellfish is extremely difficult, there has been very little research on these compounds or searches for novel brevetoxins.
Non patent document 1: McFarren E F, Silva F J, Tanabe H, Wilson W B, Campbell J E, Lewis K H . . . occurrence of a ciguatera-like poison in oysters, clams, and Gymnodinium breve cultures. Toxicon. 1965 November;3(2):111-23.
Non patent document 2: Morris P D, Campbell D S, Taylor T J, Freeman J I. Clinical and epidemiological features of neurotoxic shellfish poisoning in North Carolina. Am J Public Health. 1991 April;81(4):471-4.
Non patent document 3: Ishida H, Nozawa A, Totoribe K, Muramatsu N, Nukaya H, Tsuji K, Yamaguchi K, Yasumoto T, Kaspar H, Berkett N, Tetrahedron Letters, Vol. 36, No. 5, pp. 725-728 (1995)
Non patent document 4: Nozawa A, Tsuji K, Ishida H. Implication of brevetoxin B1 and PbTx-3 in neurotoxic shellfish poisoning in New Zealand by isolation and quantitative determination with liquid chromatography-tandem mass spectrometry. Toxicon. 2003 July;42(1):91-103.
Non patent document 5: Ishida H, Tsuji K, Sea food poisoning caused by brevetoxins in New Zealand MycToxin, No. 48, pp. 2931(1999)
Non patent document 6: Ishida H, Muramatsu N, Nukaya H, Kosuge T, Tsuji K. Study on neurotoxic shellfish poisoning involving the oyster, Crassostrea gigas, in New Zealand. Toxicon. 1996 September;34(9):1050-3.

### DISCLOSURE OF THE INVENTION

The present invention provides a shellfish neurotoxin in the form of a novel brevetoxin derivative.

The present invention relates to a compound represented by formula (I):

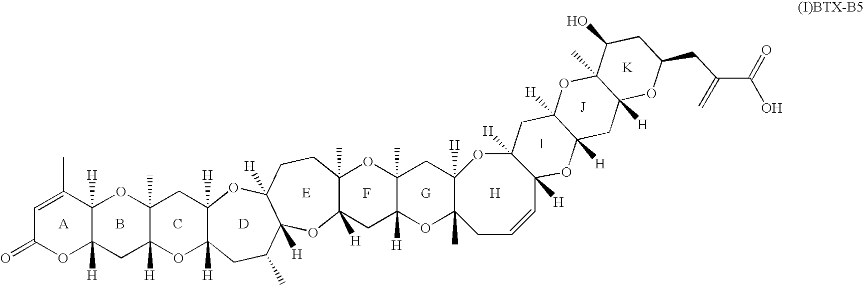

The present invention relates to a production process of a compound represented by formula (I) comprising oxidizing the aldehyde group located on the end of the compound represented by formula (II): 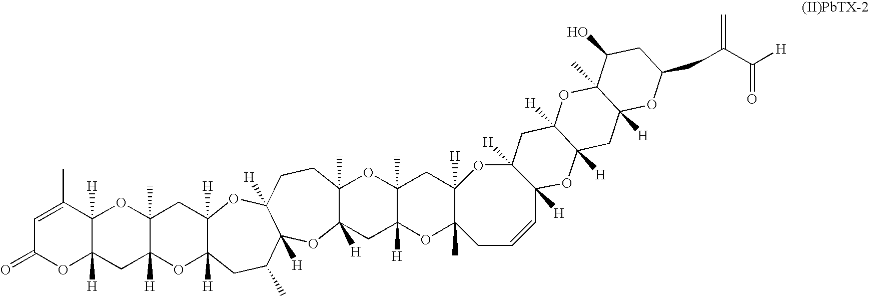
to a carboxyl group.

Moreover, the present invention relates to a method for detecting a shellfish neurotoxin in shellfish comprising quantifying the compound represented by formula (I) present in shellfish, and using said compound as a shellfish neurotoxin marker.

### EFFECT OF INVENTION

The present invention is able to provide a novel brevetoxin compound (brevetoxin B5 (BTX-B5)) that is frequently found in comparatively numerous types of shellfish as compared with other brevetoxins, and is comparatively stable. The present invention also enables a shellfish neurotoxin in shellfish to be detected by using the novel brevetoxin compound.

### BRIEF DESCRIPTION OF THE DRAWINGS

FIG. 1 shows the structures of brevetoxins PbTx-2 and PbTx-3 along with their analog, BTX-B.

FIG. 2 shows NMR techniques used for determining the structure of BTX-B5 (1)(a), the configuration of its carbon and hydrogen atoms (b), and the chemical shifts of the carbon and hydrogen atoms of BTX-B1 (2). (a) Heavy lines and arrows indicate the partial structures assigned by ¹H—¹H COSY and HMBC, respectively. (b) The attached numbers denote ¹³C NMR (¹H NMR) chemical shifts and the ppm in CD₃OD, while the arrows indicate the NOE values measured by NOE difference experiments around ether linkages.

FIG. 3 shows the negative FAB CD MS/MS spectrum of BTX-B5 along with a molecular ion at m/z 909.5 as a precursor.

FIG. 4A shows a selected reaction monitoring (SRM) liquid chromatography-tandem mass spectrometry chromatogram for 10 μl of BTX-B2 standards (50 ng/mL). (Precursor-product ion combinations and polarity used in SRM detection are shown. Vertical axis: Intensity (×10⁴) Horizontal axis: Retention time (min).

FIG. 4B shows a selected reaction monitoring (SRM) liquid chromatography-tandem mass spectrometry chromatogram for 10 μl of 80% MeOH fractions from 80% MeOH extracts of toxic cockles (g/mL). Precursor-product ion combinations and polarity used in SRM detection are shown. Vertical axis: Intensity (×10⁴). Horizontal axis: Retention time (min).

FIG. 4C shows a selected reaction monitoring (SRM) liquid chromatography-tandem mass spectrometry chromatogram for 10 μl of 80% MeOH fractions from 80% MeOH extracts of toxic greenshell mussels (g/mL). Precursor-product ion combinations and polarity used in SRM detection are shown. Vertical axis: Intensity (×10⁴) Horizontal axis: Retention time (min).

FIG. 4D shows a selected reaction monitoring (SRM) liquid chromatography-tandem mass spectrometry chromatogram for 10 μl of 80% MeOH fractions from 80% MeOH extracts of toxic Pacific oysters (g/mL). Precursor-product ion combinations and polarity used in SRM detection are shown. Vertical axis: Intensity (×10⁴) Horizontal axis: Retention time (min).

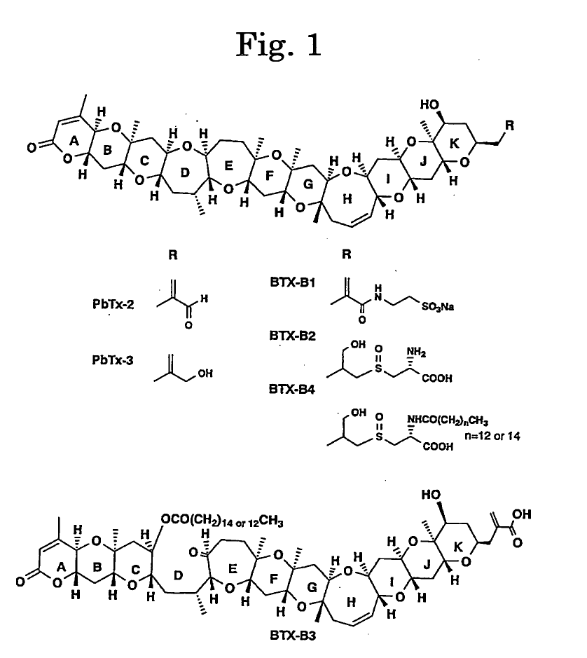

### BEST MODE FOR CARRYING OUT THE INVENTION

A compound represented by formula (I) of the present invention can be extracted and isolated from shellfish as described in the following examples.

A compound represented by formula (I) can also be synthesized by using, for example, an oxidizing agent, to oxidize the aldehyde group on the end of the compound represented by formula (II) to a carboxyl group. The compound represented by formula (II) is commonly known as PbTx-2, and can be prepared according to the process described in Lin Y. Y, Risk M, Rays M, Eugen D V, Clardy J, Golik J, James J C, Nakanishi K, J. Am. Chem. Soc., 81, 6773-6775 (1981).

Examples of oxidizing agents used include those capable of being used to oxidize aldehydes, and hydrogen peroxide is used preferably.

The oxidation reaction is preferably carried out in the presence of a catalyst, an ordinary oxidation catalyst can be used, and SeO₂ is used preferably.

The oxidation reaction can be preferably carried out under the conditions indicated below.
Reaction temperature: 0 to 50° C. (particularly preferably 5 to 45° C., and more preferably 10 to 40° C.; room temperature)
Reaction pressure: 0.1 to 10 MPa (and particularly preferably 0.5 to 5 MPa; normal pressure)
Solvent: Organic polar solvent (lower alcohol-based organic solvent such as i-butanol)
Catalyst: Oxidation catalyst such as SeO₂
Oxidizing agent: Oxidizing agent such as hydrogen peroxide

Since the compound of the present invention (BTX-B5) is frequently found in comparatively numerous types of shellfish contaminated with brevetoxin as compared with other brevetoxins and is comparatively stable, the degree of contamination by shellfish neurotoxin can be determined based on confirmation of the presence of this compound and quantification thereof. Accordingly, this compound can be used as a intoxication marker of shellfish neurotoxin to enable detection of shellfish neurotoxin in shellfish.

### EXAMPLE 1

### Purification and Isolation of BTX-B5

Toxic cockles (160 kg) obtained in Whangerei, New Zealand were shucked, freeze-dried in a vacuum and ground followed by extracting twice with 80% methanol while refluxing (heating). The extract was partitioned between CH₂Cl₂ and H₂O. The organic layer was further partitioned between n-hexane and 80% methanol. The methanol layer (109.2 g) was then subjected to SiO₂ chromatographic separation using CH₂Cl₂—MeOH—H₂O (95:5:0, 65:15:2, 65:20:3 and 65:45:10) to obtain four fractions consisting of fr1-1 to fr1-4, respectively. Fractions fr1-1, fr1-2 and fr1-3 demonstrated neurotoxicity in mice assay. The mouse assay were carried out in the manner described below. Each test sample was suspended in 1% Tween60-physiological saline, and administered intraperitoneally to ddY mice weighing 18 to 22 g. Mice were observed continuously for six hour and hourly from six to fifth hour. Mouse unit (MU) toxicity was extrapolated from the dosage-response chart indicated in the official protocol (Delaney, J. E., 1985. Chapter 4: Bioassay procedures for shellfish toxins. In: Greenburg, Burg (Eds.). Laboratory procedures for the examination of sea water and shellfish, fifth ed., American Public Health Association Inc., Washington, D.C. pp. 66-78). The MU of BTX-B1 and PbTx-3 were converted using conversion coefficients of 3.6 and 4.0 μg/MU, respectively, based on detection data in mouse.

An fr2-6 fraction was obtained from fr2-1 using ODS-A₆₀ (YMC-Gel) based on fr1-2 and fr1-3. The two fractions fr2-5 and fr2-6 that eluted using 70% and 80% MeOH demonstrated activity. Next, fr2-5 was chromatographed using Sephadex LH-20 and finally applied to YMC ODS-A₃₂₄ column chromatography to obtain BTX-B1 (12 mg).

Fraction fr1-1 was further purified by successively chromatographing on Sephadex LH-20 using MeOH, on SepPak C₁₈ using 80% MeOH, on Puresil (Millipore) C₁₈ using 85% MeOH and finally on LiChroCART RP-18 (Merck) using 80% MeOH to obtain PbTx-3 (approximately 2.4 mg).

Fraction fr2-6 was further purified by successively chromatographing on Sephadex LH-20 using MeOH, on Puresil C₁₈ (×2) using 80% MeOH, and finally on LiChroCART RP-18 using 80% MeOH to obtain brevetoxin B5 (BTX-B5) (approximately 500 μg). Each eluate was investigated for the presence of toxic activity by mice assay.

### Example 2

### Structural Analysis of BTX-B5

The toxic BTX-B5 obtained in Example 1 was in the form of a colorless amorphous solid that demonstrated the physical properties described below.
FABMS m/z negative, 909 (M−H)⁻; positive, 933 (M+Na)⁺
HR-FBMS, m/z 933.4645 (M+Na)⁺ (calculated value for C₅₀H₇₀O₁₅, 933.4642)

The infrared absorptions with KBr tablets at 3446, 1735, 1652, 1609, 1230, 1211 and 865 cm⁻¹ suggested the presence of a hydroxyl group, two conjugated carboxylates and a carboxyl functional group in the molecule.

Similar to PbTx-2 and BTX-B1, since this compound has conjugated carboxylates and a carboxylic acid chromophore group, it has an absorbance maximum (absorption peak) at (UV)λmax (MeOH) of 205 nm (ε27,300) in the ultraviolet spectrum.

Although the 1D proton NMR (CD₃OD) spectrum of BTX-B5 resembles that of PbTx-2, it does not have an aldehyde signal, and is substantially identical to that of BTX-B1 with the exception of signals resulting from the taurine group of the side chain.

According to the results of ¹H—¹H COSY measurement, good agreement was demonstrated in the connectivities, chemical shifts and coupling constants of protons from H2 to H40 between BTX-5 and BTX-B1(2). On the basis of these findings, BTX-5 is similar to BTX-B1, and has the same polyester portion and stereochemistry.

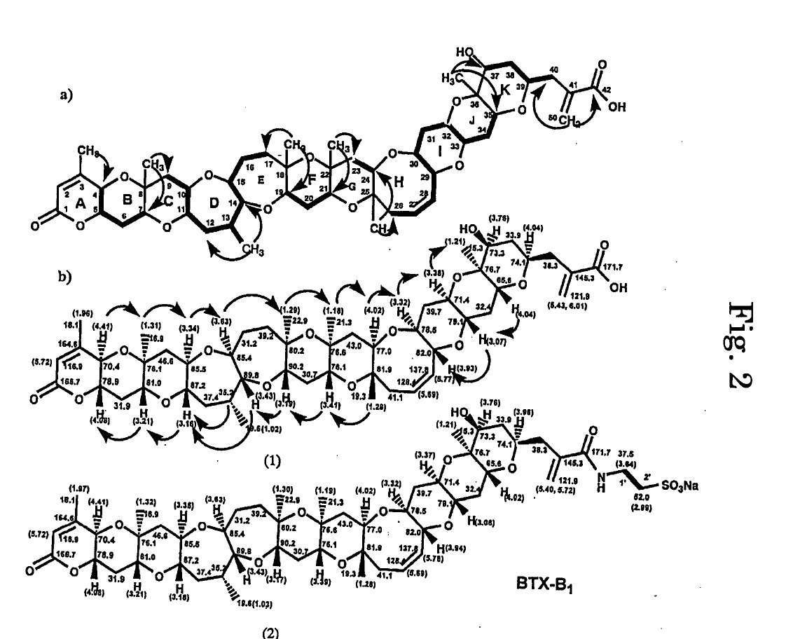

C40-C41(C50)-C42 was confirmed based on allyl coupling observed between H50 and H40, and HMBC correlation between H50 and C42. Accordingly, the functional group at C42 in the side chain of BTX-B5 is —COOH (FIG. 2).

The CD spectrum of BTX-B5 (MeOH) demonstrated a negative maximum (Δε −5.78 ene-lactone ππ*) at 227 nm and a positive maximum (Δε +6.88 ene-lactone nπ*) at 257 nm, and these were similar to those found in BTX-B1 and PbTx-2. These results indicate that BTX-B5 has the same absolute configuration as PbTX-2.

The proposed structural formula was well supported by collisionally-activated dissociation, negative ion FAB MS/MS experiments carried out on the (M−H)− ion ((m/z 909) of BTX-B5.

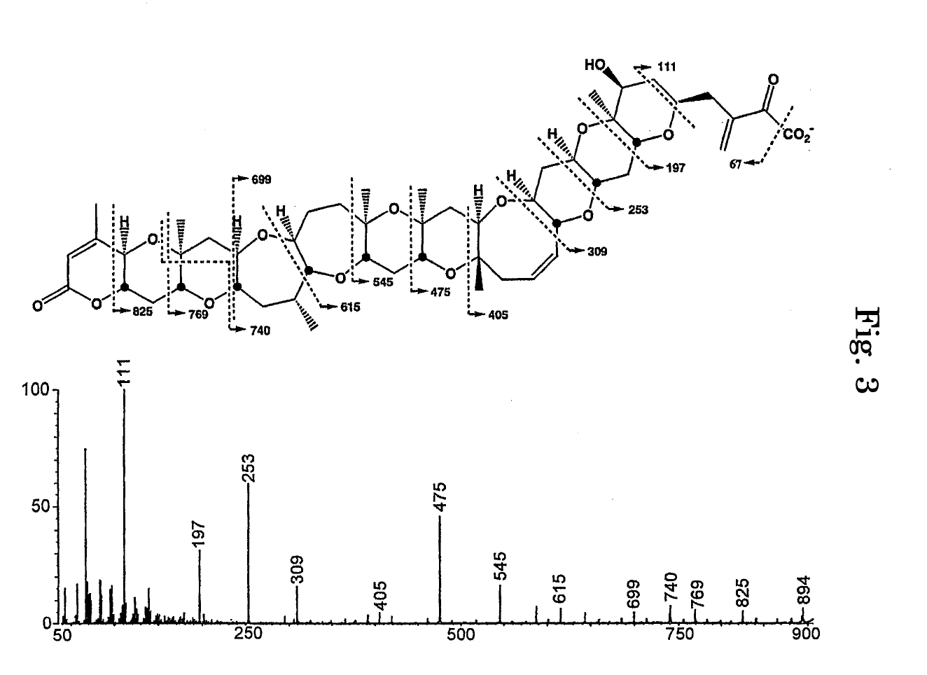

Bond cleavage between C37-C36 and C39-O was verified by generation of ion m/z 111 (FIG. 3). Other prominent ions were generated by characteristic bond cleavage at ether rings, as observed in reported BTX-B2 (Murata K, Satake M, Naoki H, Kaspar H F, Yasumoto T, Tetrahedron 54, 735-742 (1989)), and were consistent with the proposed structural formula.

### EXAMPLE 3

### Synthesis of BTX-B5

1) 30% H₂O₂ (0.5 ml) was gradually added while stirring to a solution in which PbTx-2 (1.0 mg) was dissolved in t-BuOH (1 ml) containing SeO₂ (0.5 mg) at room temperature. After diluting the reaction solution with water (9 ml) 4 hours after adding the 30% H₂O₂, the diluted reaction solution was applied to solid-phase column Oasis HLB plus cartridges (manufactured by Waters). After washing with 70% methanol (5 ml), the product was eluted with 90% methanol. Approximately 0.9 mg of BTX-B5 were obtained by this method.

2) Synthesis of BTX-B5

5% NaClO₂ (1.0 ml) was gradually added while stirring to a solution in which PbTx-2 (1.0 mg) was dissolved in methanol (1 ml) at room temperature. After diluting the reaction solution with water (9 ml) 4 hours after adding the 5% NaClO₂, the diluted reaction solution was applied to solid-phase column Oasis HLB plus cartridges (Waters). After washing with 70% methanol (5 ml), the product was eluted with 90% methanol. Approximately 0.9 mg of BTX-B5 were obtained by this method.

### EXAMPLE 4

### Quantification of BTX-B5

HPLC conditions; column: Cadenza CD-C18 (3 mm×150 mm, 3 μm), mobile phase: gradient of 0.1% formic acid-acetonitrile (20-80% acetonitrile) for 20 minutes, flow rate: 0.2 mL/min, 20 μL injection. Cone voltage was set to 100 V and collision-induced dissociation was carried out at a collision energy of 75 eV. Argon was used for the collision gas.

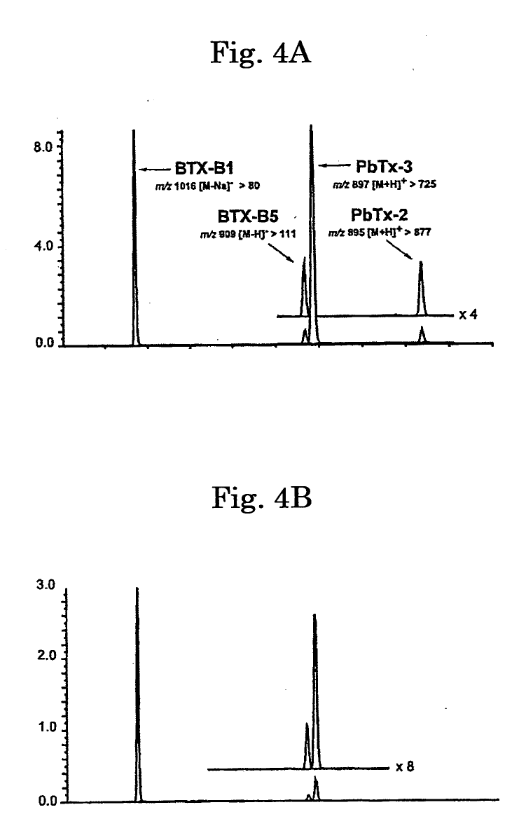

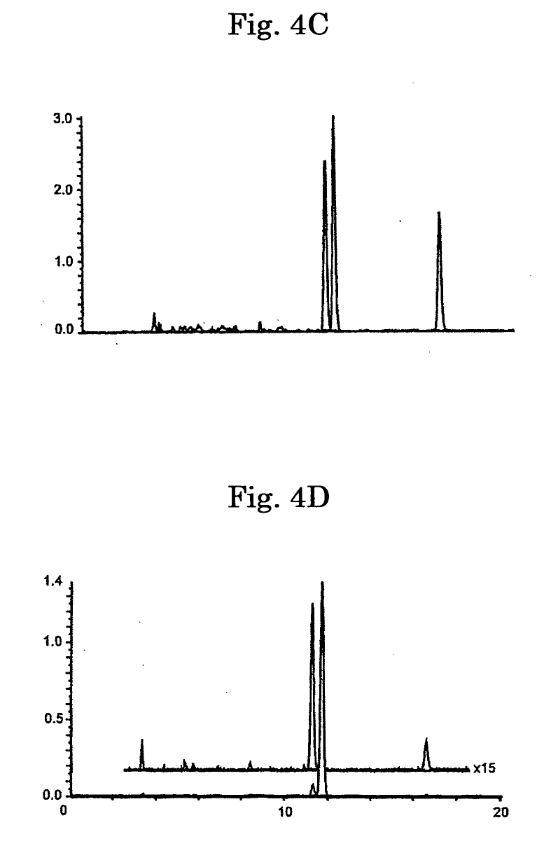

BTX-B5 along with PbTx-3 were found in the New Zealand toxic shellfish, cockle, greenshell mussel (Perna (P.) conaliculus) and Pacific oyster, which had been harvested at an outbreak of NSP early in 1993, by liquid chromatography-tandem mass spectrometry. On the other hand, high and low levels of BTX-B1 were confirmed in cockle and Pacific oyster, respectively (FIG. 4).

The minimum lethal dose of BTX-B5 by intraperitoneal administration isolated in the manner previously described was approximately 0.3 to 0.5 mg/kg. Immediately after injection, the test animals demonstrated symptoms of neurotoxicity extremely similar to those caused by other brevetoxins.

Interestingly, although it is well known that several brevetoxins such as PbTx-1, PbTx-2 and PbTx-3 are produced in the approximate ratio of 1/7/2 by the dinoflagellate G. breve, the potent ichthyotoxins PbTx-1 and Pb-Tx2 were not detected at significant levels in cockles, while the less ichthyotoxic and lethal BTX-B3, BTX-B1 and BTX-B5 were found in the ratio of approximately 5/25/1.

Very recently high concentrations of PbTx-3 and BTX-B5 were detected in the same greenshell mussels from New Zealand. On the basis of this analytical data, it was shown that PbTx-3, BTX-B5 and BTX-B1 are responsible for the NSP-associated toxicity of cockles, while PbTx-3, BTX-B5, BTX-B2, BTX-B3 and BTX-B4 are responsible for that toxicity in greenshell mussels, and PbTx-3 and BTX-5 are responsible for that toxicity in Pacific oysters.

In this manner, BTX-B5 and PbTx-3 are able to serve as excellent markers for monitoring shellfish toxicity following G. breve algal bloom. Since BTX-B2, BTX-B3 and BTX-B4 are only obtained from cockles, brevetoxin metabolism was suggested to be species-specific.

Brevetoxins, and particularly PbTx-1 and PbTx-2, have been reported to be unstable under acidic and basic conditions. It was newly found that the terminal aldehyde group of PbTx-2 is readily converted to the corresponding carboxyl group by selective oxidation under mild conditions. Although it is believed that PbTx-2 is metabolized to BTX-B5 in all three of these shellfish for detoxification, and that the majority of the BTX-B5 is consumed by being converted to BTX-B1 by internal cockle enzymes, the conversion rates by these enzymes in greenshell mussel and Pacific oyster are extremely low. Since this metabolism resembles the aldehyde detoxification pathway in the human body, elucidation of the brevetoxin detoxification pathway in cockles that we have proposed is believed to provide beneficial findings for the treatment of NSP in humans.

It will be necessary to elucidate the mechanism and enzymes involved in the metabolism of brevetoxins in greater detail.

Highly sensitive methods utilizing liquid chromatography coupled with tandem mass spectrometry techniques for brevetoxins and their analogs will be important for shellfish toxin monitoring and metabolic studies.

### INDUSTRIAL APPLICABILITY

Since a novel compound (BTX-B5), which is one of the brevetoxin analogs in the present invention, can be used as a toxicity marker when investigating the presence of neurotoxicity in shellfish, it can be used to monitor shellfish toxicity by food companies, fishing cooperatives and so forth in the field of fisheries and marine products.

1. A compound represented by formula (I):

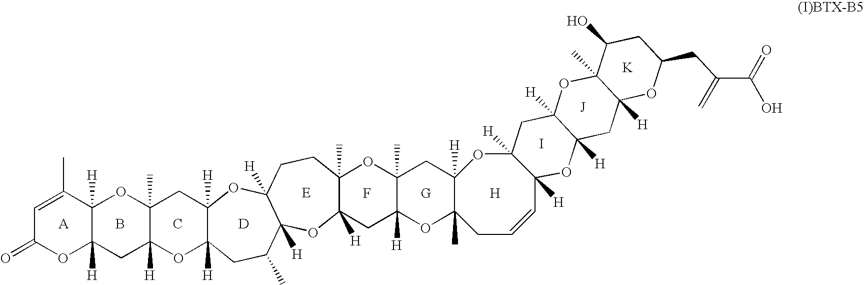

2. A production process of a compound according to claim 1 represented by formula (I) comprising oxidizing the aldehyde group located on the end of the compound represented by formula (II): 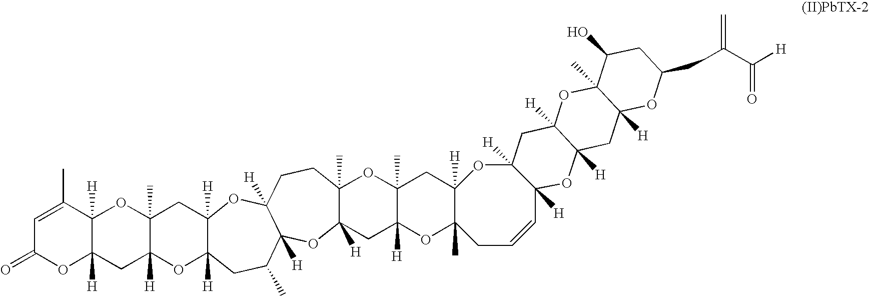
to a carboxyl group.

3. A method for detecting a shellfish neurotoxin in shellfish comprising quantifying the compound according to claim 1 represented by formula (I) present in shellfish, and using said compound as a shellfish neurotoxin marker.
